(19) United States
(12) Patent Application Publication
(10) Pub. No.: US 2019/0143284 A1
(43) Pub. Date: May 16, 2019

(54) SHAKING SYSTEM OF CELL CULTURE AND METHOD FOR SHAKING THE SAME

(71) Applicant: INDUSTRIAL TECHNOLOGY RESEARCH INSTITUTE, Hsin-Chu (TW)

(72) Inventors: Yuan-Fa LEE, Tainan City (TW); Shun-Sheng KO, Kaohsiung City (TW); Yen-Bor FANG, Pingtung County (TW); Chih-Hung HUANG, Chiayi City (TW); Li-Wen LAI, Tainan City (TW); Chih-Ching LIAO, Hsinchu County (TW)

(73) Assignee: INDUSTRIAL TECHNOLOGY RESEARCH INSTITUTE, Hsin-Chu (TW)

(21) Appl. No.: 15/922,163

(22) Filed: Mar. 15, 2018

(30) Foreign Application Priority Data
Nov. 10, 2017 (TW) 106139031

(51) Int. Cl. B01F 11/00; C12M 3/06; C12M 3/04; C12M 1/34

(52) U.S. Cl. CPC B01F 11/0097; C12M 27/16; C12M 27/10; B01F 11/0008; C12M 41/32; B01F 11/0014; C12M 41/44; B01F 11/0048

(57) ABSTRACT

A shaking system of cell culture includes a shaking unit, a side visual unit, a top visual unit and a control unit. The side visual unit is located at one side of the shaking unit. The top visual unit is located above the shaking unit. The control unit is coupled signally with the shaking unit, the side visual and the top visual unit. In addition, a shaking method of cell culture is provided as well.

### CROSS REFERENCE TO RELATED APPLICATION

This application claims the benefits of Taiwan application Serial No. 106139031 filed on Nov. 10, 2017, the disclosures of which are incorporated by references herein in its entirety.

### TECHNICAL FIELD

The present disclosure relates in general to a shaking system of cell culture and a method thereof.

### BACKGROUND

In the art of cell culture, the culture medium should be replaced periodically so as to assure a well cell-growth environment. While in replacing the culture medium, the culture fluid having the culture medium shall be shaken well by shaking so as to distribute thoroughly and evenly over cells to be cultivated.

Sometimes, the aforesaid shaking of the culture fluid would meet a coverage problem of the culture medium over the cells, and also some culture fluid may be occasionally swung high to the mouth of the cell-culture dish such that a contamination of the culture fluid is highly possible. Thereupon, the cells to be cultivated won't grow well.

Accordingly, efforts to improve the coverage of the culture medium and to prevent the culture fluid from being swung to touch the mouth of the cell-culture dish are definitely urgent to the skill in the art.

### SUMMARY

In this disclosure, an shaking system of cell culture includes:

an shaking unit;

a side visual unit, located at one side of the shaking unit;

a top visual unit, located above the shaking unit; and

a control unit, coupled signally with the shaking unit, the side visual unit and the top visual unit.

In this disclosure, an shaking method of cell culture includes the steps of:

Shaking a cell-culture dish, which is furnished in a shaking system and contains a kind of culture fluid, at a fixed rotation speed within a small angular range;

Acquiring an image information of the cell-culture dish by a top visual unit;

Acquiring a distribution area of the culture fluid by a control unit in accordance with the image information;

Performing rotationally shaking toward a non-liquid surface distribution area in accordance with the distribution area,

Changing an shaking speed and an shaking angle by the shaking system to make the culture fluid distributed in the non-liquid surface distribution area;

Acquiring the image information of a culture liquid surface height of the cell-culture dish by the side visual unit;

Changing the shaking angle by the control unit in accordance with the image information of the liquid surface height to increase or decrease the shaking angle;

Judging if a 100% culture medium coverage has been achieved;

Acquiring again the image information of the cell-culture dish by the top visual unit and the side visual unit; and,

Confirming a distribution status of the culture liquid by the control unit in accordance with an image information history.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a schematic view of an embodiment of the shaking system of cell culture in accordance with this disclosure;

FIG. 2 is a schematic perspective view of the shaking unit of FIG. 1;

FIG. 3 is a schematic perspective view of the variable eccentric-rotating module of FIG. 2;

FIG. 4 is a schematic cross-sectional view of FIG. 3;

FIG. 5 is a schematic perspective view of the linkage module of FIG. 2;

FIG. 6 is a schematic cross-sectional view of FIG. 5;

FIG. 7 is a schematic perspective view of the variable center-rotating module of FIG. 2;

FIG. 8 is a schematic cross-sectional view of FIG. 7;

FIG. 9 is a flowchart of an embodiment of the shaking method of cell culture in accordance with this disclosure;

FIG. 10 is schematic plot of position changes of the rotational center point in accordance with the this disclosure;

FIG. 11 is a schematic view of position changes of the rotational center point of the variable1 center-rotating module in accordance with this disclosure;

FIG. 12 is a schematic view of position changes of the shaking unit in accordance with this disclosure;

FIG. 13 is another schematic view of position changes of the shaking unit in accordance with this disclosure;

FIG. 14 shows schematically the shaking unit at a first shaking angle; and

FIG. 15 shows schematically the shaking unit at a second angle.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

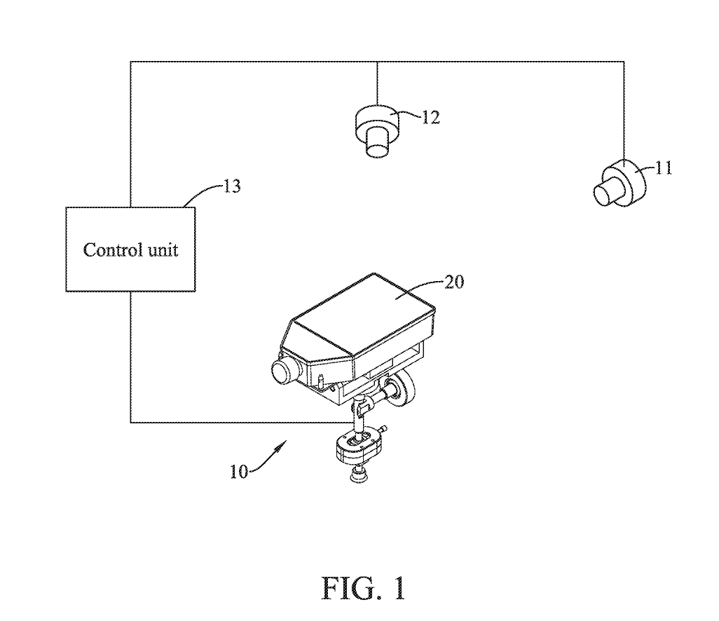

Referring now to FIG. 1, the shaking system of cell culture includes a shaking unit 10, a side visual unit 11, a top visual unit 12 and a control unit 13.

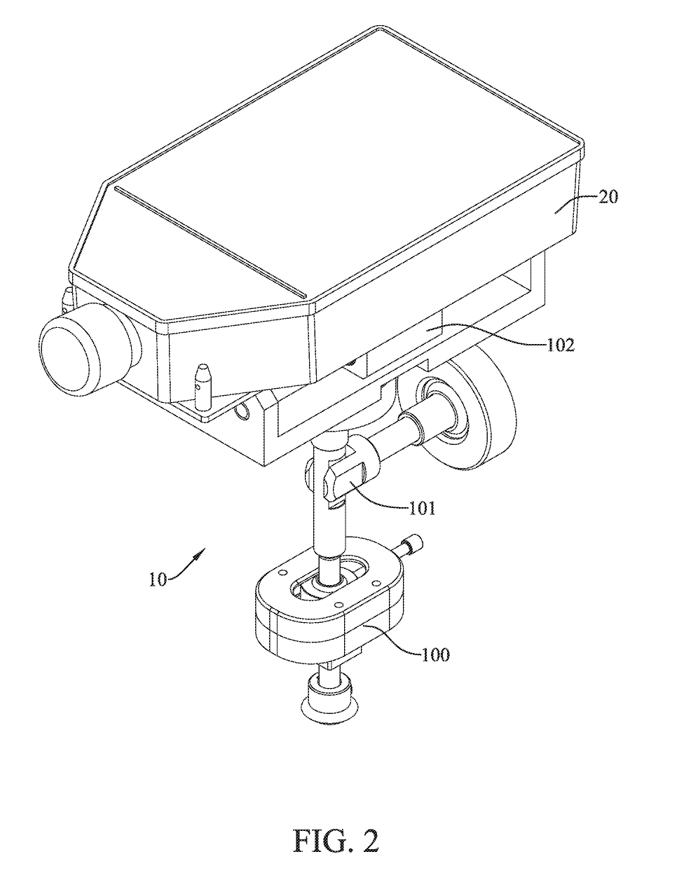

Referring now to FIG. 2, the shaking unit 10, connected signally to the control unit 13, has a variable eccentric-rotating module 100, a linkage module 101 and a variable center-rotating module 102.

As shown in FIG. 1 and FIG. 2, a cell-culture dish 20 is arranged fixedly on the variable center-rotating module 102.

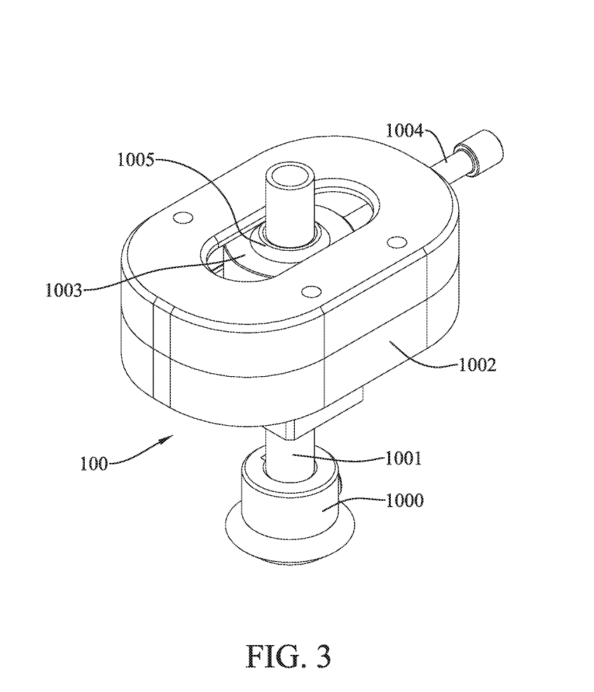

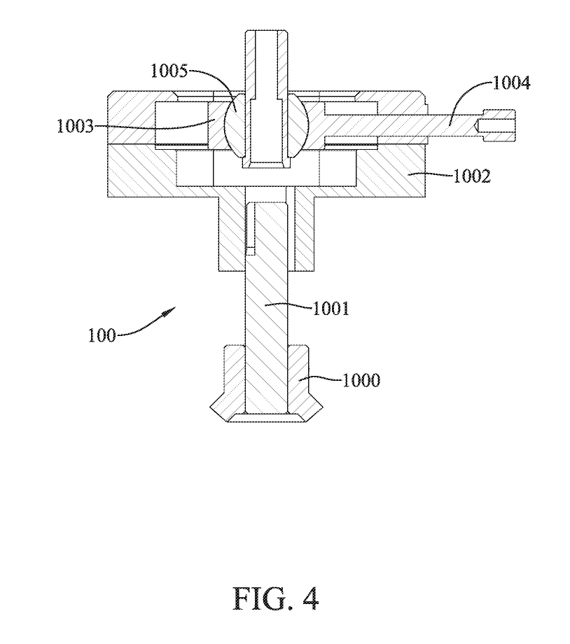

Referring now to FIG. 3 and FIG. 4, the variable eccentric-rotating module 100 has a drive gear 1000, a transmission shaft 1001, an eccentric cam 1002, an eccentric slider 1003, an adjusting member 1004 and a first spherical bearing 1005.

The drive gear 1000 is coupled with a power source. The power source can be a motor, a gear set, a screw bar, a pneumatic cylinder or a hydraulic cylinder. The transmission shaft 1001 is coupled, by one end thereof, to and thus driven by the drive gear 1000.

The eccentric cam 1002 for providing an inner cam contour is coupled to and thus driven by the transmission shaft 1001. The eccentric slider 1003 is mounted inside the eccentric cam 1002 so as to contact the inner cam contour. The adjusting member 2004 is connected to the eccentric slider 1003 so as able to adjust a position of the eccentric slider 1003 with respect to the eccentric cam 1002; i.e., an eccentric position of the eccentric slider 1003 to further vary a shaking angle of the shaking unit 10.

The first spherical bearing 1005 is coupled with the eccentric slider 1003 so as thereby to overcome possible angular problem while in performing the aforesaid eccentric movement.

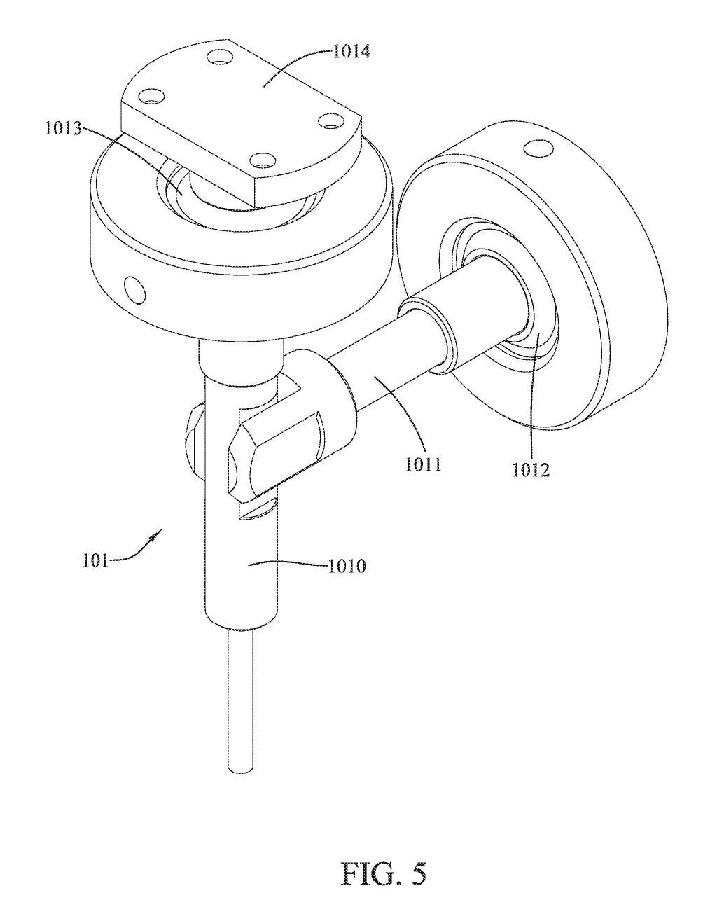

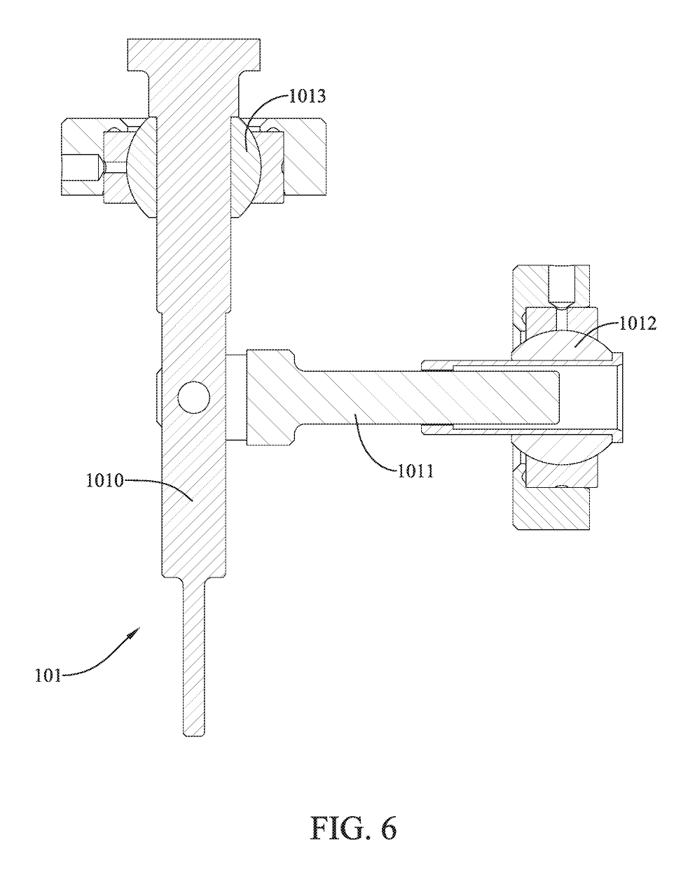

Referring now to FIG. 5 and FIG. 6, the linkage module 101, coupled with the variable eccentric-rotating module 100, includes a connecting rod 1010, an auxiliary guide bar 1011, a second spherical bearing 1012 and a third spherical bearing 1013.

One end (the lower end in the figure) of the connecting rod 1010 is coupled to the first spherical bearing 1005. One end of the auxiliary guide bar 1011 is connected to a middle point of the connecting rod 1010, while another end thereof is connected to the second spherical bearing 1012. The second spherical bearing 1012 is mounted by a grounded installation surface. While the shaking unit 10 rotates, the second spherical bearing 1012 and the auxiliary guide rod 1011 can prevent the connecting rod 1010 from revolutions. The third spherical bearing 1013 is connected with another end of the connecting rod 1010.

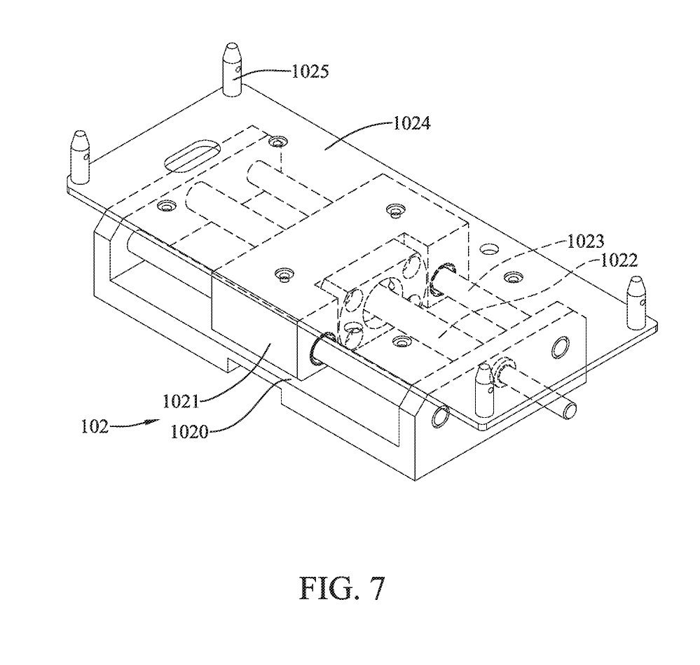

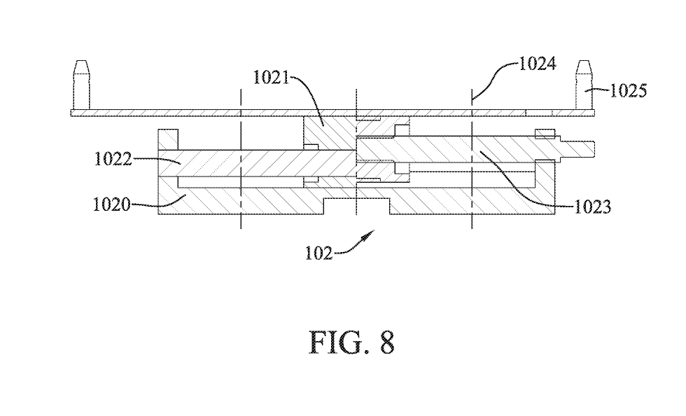

Referring now to FIG. 7 and FIG. 8, the variable center-rotating module 102, coupled with the linkage module 101, has a base frame 1020, a slider 1021, at least one guide bar 1022, a screw bar 1023 and a carrier board 1024.

The base frame 1020 is coupled with the third spherical bearing 1013, which is served as a pivotal point of the shaking unit 10. As shown, the base frame 1020 provides two upright side walls opposing to each other so as to define an inner space for the slider 1021 to move back and forth.

The slider 1021, mounted on the base frame 1020 by locating between the two side walls, can displace with respect to the base frame 1020. The guide bar 1022 and the screw bar 1023 are individually sent through both the base frame 1020 (at the two side walls) and the slider 1021. The screw bar 1023, further coupled with a power source, drives the slider 1021 to displace on the base frame 1020. Thereupon, the rotational center point of the variable center-rotating module 102 can be varied. The carrier board 1024, connected fixed with the slider 1021, has four corners, where each of the four corners is furnished with an individual positioning pin 1025 for locating a cell-culture dish 20 on the carrier board 1024.

Referring also to FIG. 1, the top visual unit 12, located above the shaking unit 10, is to capture top-viewed image information of the cell-culture dish 20 fixed on the carrier board 1024. The top visual unit 12 is coupled signally to the control unit 13.

The side visual unit 11, located at a side of the variable center-rotating module 102 of the shaking unit 10, is to capture side-viewed image information of the cell-culture dish 20 fixed on the carrier board 1024. The side visual unit 11 is coupled signally to the control unit 13. In this disclosure, the top visual unit 12 and the side visual unit 11 are both charge coupled devices (CCD), digital cameras, or digital recorders.

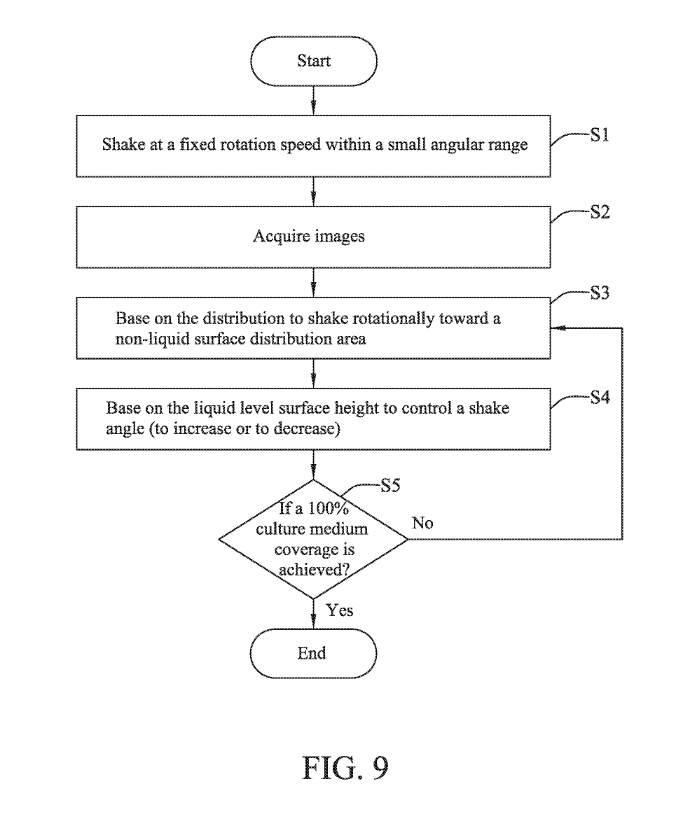

Referring now to FIG. 9, a shaking method of cell culture in accordance with this disclosure can include the following steps.

In Step S1, shake a cell-culture dish by a fixed rotation speed within a small angular range. As shown in FIG. 1 and FIG. 2, the cell-culture dish 20, arranged in the shaking system of cell culture, has a culture fluid. The power source drives the variable eccentric-rotating module 100 by the fixed rotation speed. The variable eccentric-rotating module 100 then drives the linkage module 101. Further, the linkage module 101 drives the variable center-rotating module 102. In an exemplary example of this disclosure, the variable eccentric-rotating module 100 is to drive the variable center-rotating module 102 to shake within the small angular range. In this embodiment, the fixed rotation speed can be 9.5 rpm, and the small angular range is ±50°.

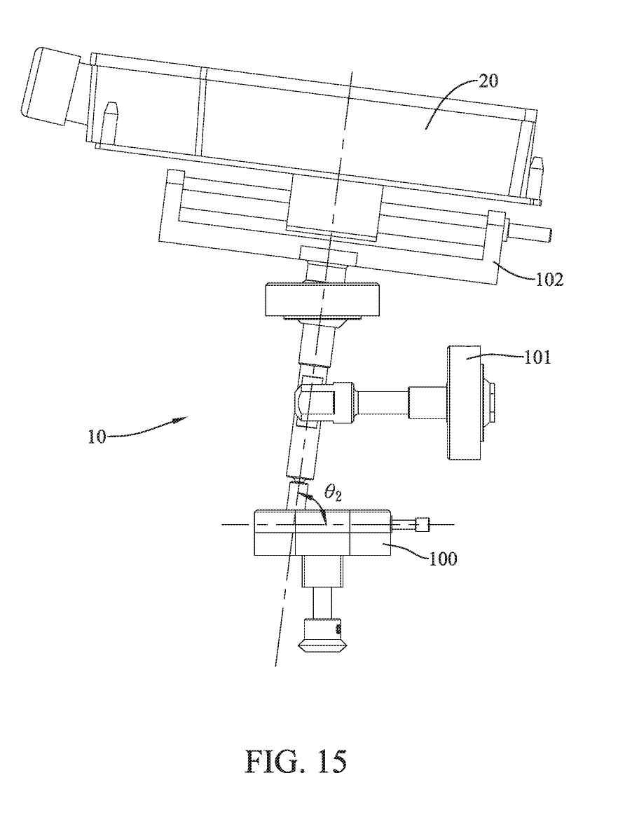

Referring to FIG. 3, in this disclosure, a differential ratio of the drive gear 1000 to the power source is applied to achieve the fixed rotation speed of 9.5 rpm. In addition, referring to FIG. 4, FIG. 15 and FIG. 16, the small angular range for the shaking of the variable center-rotating module 102 can be determined by the position of the eccentric slider 1003 with respect to the eccentric cam 1002.

In this Step 1, in order not to jeopardize the growth of cells by an excessive shear stress resulted from a high-speed shaking or a compression by bubbles generated from an abrupt change in rotation speed, thus the fixed rotation speed and the small angular range are initially applied to shake the cell-culture dish 20. Thereupon, the liquid level of the culture fluid in the cell-culture dish 20 won't be swung high to the mouth of the cell-culture dish 20, and thus a risk of contaminating the culture medium can be avoided.

Step S2, capture an image. The top visual unit 12 and the side visual unit 11 are applied to capture image information (including the aforesaid top-viewed image information and the aforesaid side-viewed image information, respectively) of the cell-culture dish 20 positioned in the shaking system of cell culture. In this embodiment, the image information includes a distribution of the culture fluid (in the top-viewed image information) and a shake height of the liquid level of the culture fluid (in the side-viewed image information).

The control unit 13 receives the image information, and determines a region of interest (ROI) from the image information. Then, the region of interest is transformed into a corresponding gray-scale image. The gray-scale image is further strengthened and transformed into a binarized image. Further, a noise-filtration process is performed upon the binarized image so as to form a processed image for calculating the distribution of the culture fluid. Similarly, the shake height of the liquid level (i.e., the liquid level height) can be also obtained by the aforesaid procedures.

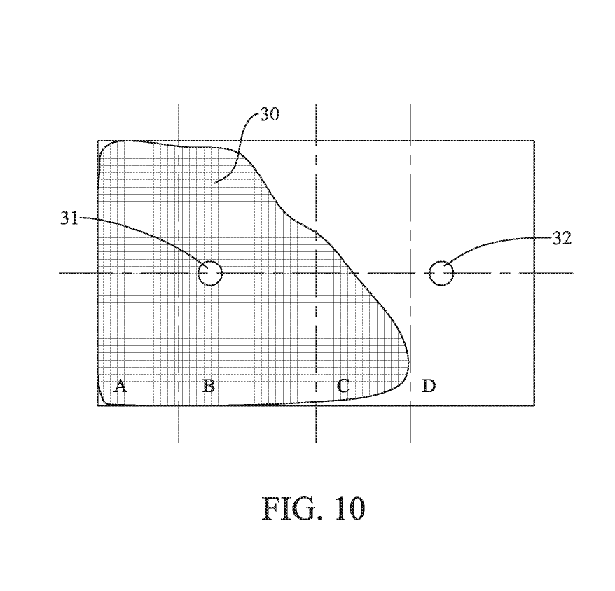

For example, referring to FIG. 10, a grid area 30 is used to stand for a major distribution area of the culture fluid. The image information captured by the top visual unit 12 is provided to the control unit 13 for determining the distribution of the culture fluid.

To calculate the liquid distribution, for example, a coordinate of the center of gravity of the covered area and the uncovered area of the culture fluid can be obtained by the following equation.

Coordinate of gravity in the distribution map=(sum (Xi)/n,sum (Yi)/n),

in which Xi and Yi are point sets of the map, and n is the number of the point sets.

Regarding the gray-scale image as described above, the control unit 13 divides the cell-culture dish 20 into a plurality of regions according to the coverage of the culture medium, and the corresponding gray-scale value of each region is recorded. These information would be used to judge the distribution uniformity of the culture fluid, by which the problem in ill coverage can be understood and thus be finally resolved. The equation for such a calculation can be as follows.

$${m}_{i} = {\sum }_{k = 1}^{n} f ( x , y ) , i = 1 \text{∼} n$$

in which f is the input image, n is the total number of the pixels, f (x, y) is the gray-scale value of the pixel at the coordinate (x, y).

Step S3, according to the distribution of the culture fluid, perform rotational shaking toward the uncovered areas. The control unit 13 bases on the instant distribution of the culture fluid obtained in Step S2 to determine the shaking toward the uncovered areas, to adjust a displacement of the eccentric slider 1003 of the variable eccentric-rotating module 100 with respect to the eccentric cam 1002, and to enlarge the angular range of shaking.

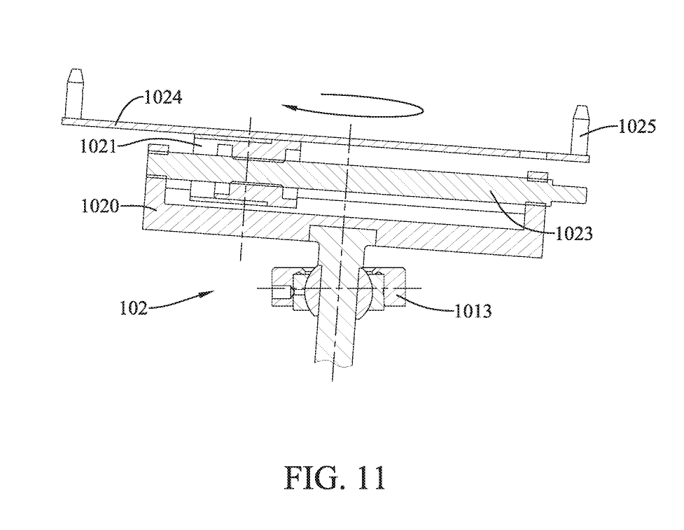

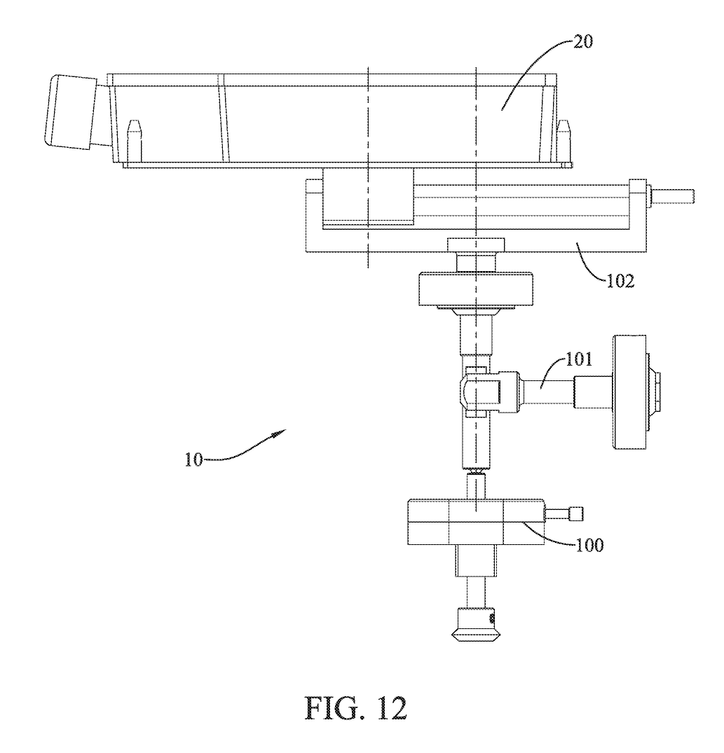

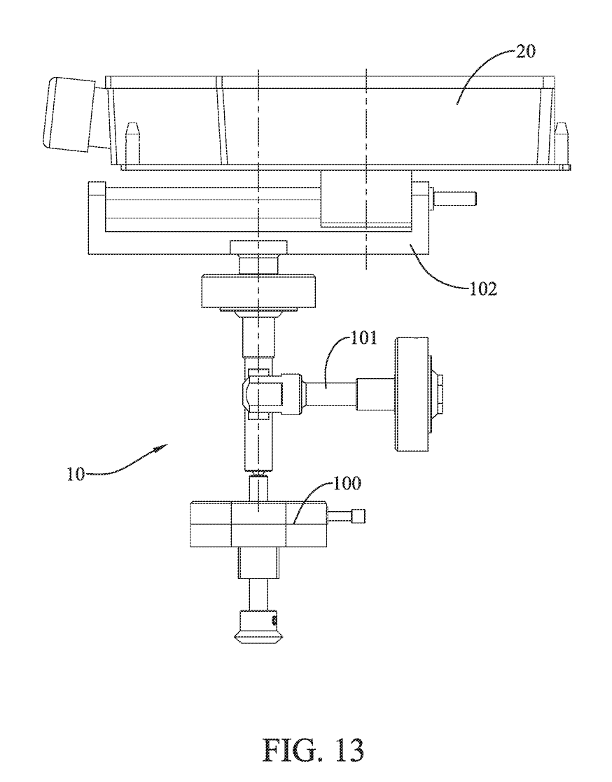

Similarly, while in enlarging the angular range of shaking, the power source of the variable center-rotating module 102 rotates the screw bar 1023 to displace linearly the slider 1021 with respect to the base frame 1020. Referring to FIG. 10 through FIG. 13, as the position of the slider 1021 is changed, the rotational center point would be shifted from the first rotation center 31 to the second rotation center 32.

By equally dividing FIG. 10 into plural image regions as an example, it is seen that FIG. 10 is divided equally into, but not limited to, four image regions; a first region A, a second region B, a third region C and a fourth region D, positioned from left to right in the figure. If the first region A is a covered region of the culture fluid and the second region B is not fully covered by the culture fluid, then the variable center-rotating module 102 would rotates the center point toward the second region B. Thus, the rotational center point can be shifted from the first rotation center 31 to the second rotation center 32.

Step S4, according to the liquid level height, control the shaking angle to increase or decrease. The side visual unit 11 firstly captures the side-viewed image information having the liquid level height of the cell-culture dish 20. Then, as described in Step S2, the control unit 13 analyzes the side-viewed image information captured by the side visual unit 11 so as thereupon to control the slider 1021 to displace with respect to the base frame 1020, such that the corresponding shaking angle can be increased or decreased (i.e., to enlarge or narrow the angular range of shaking).

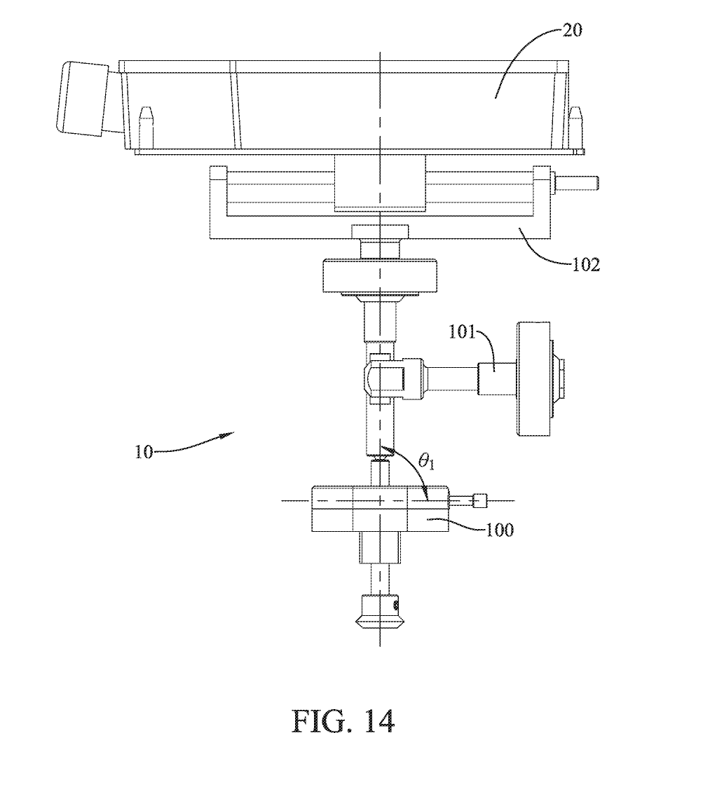

Referring to FIG. 14, the shaking unit 10 shakes the cell-culture dish 20 by a first shaking angle Θ1.

On the other hand, referring to FIG. 15, the shaking unit 10 shakes the cell-culture dish 20 by a second shaking angle Θ2.

As shown in FIG. 14 and FIG. 15, the shaking unit 10 narrows the angular range of shaking by the first shaking angle Θ1, while the shaking unit 10 enlarges the angular range of shaking by the second shaking angle Θ2.

As described in Step S2, the control unit 13 receives the side-viewed image information, then chooses an ROI from the side-viewed image information, transforms the ROI into a corresponding gray-scale image, strengthens the gray-scale image so as further to transform the gray-scale image into a corresponding binarized image, and finally performs a noise-filtration process upon the binarized image so as to obtain a corresponding processed image. The processed image is used for computing the liquid level height.

Step S5, determine whether or not a 100% culture medium coverage has been achieved. In this step, the top visual unit 12 and the side visual unit 11 capture again the image information of the cell-culture dish 20 (the top-viewed image information and the side-viewed image information, respectively). The control unit 13 bases on all changes of the individual gray-values in all the image information, instant and historical, to confirm the distribution of the culture fluid and to obtain a computed coverage of the culture fluid. Based on the computed coverage, it is determined whether or not a further shaking is necessary. If negative, then keep shaking, and go back to Step S3. If positive, then stop the shaking.

In summary, the shaking system of cell culture and the method thereof in accordance with this disclosure can provide a uniform coverage of the culture medium over the cells in the cell-culture dish and functions of monitoring and feeding back the liquid level height and the angle of the culture fluid in the cell-culture dish. Thereupon, a well environment for growing the cells can be provided.

The shaking system of cell culture and the method thereof provided by this disclosure can dynamically control the flow speed and the coverage area of the cell-culture fluid. In addition, various shake postures including pitch, roll, yaw and the like complicated shake motion can be performed to the culture fluid inside the cell-culture dish. Thus, the shaking system of cell culture and the method thereof in accordance with this disclosure can dynamically monitor the liquid level of the culture fluid, and simultaneously feed back the angle thereof. Thereupon, the culture medium can be prevented from touching the mouth of the cell-culture dish, and thus cultivation failure from contamination can be successfully avoided.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. An shaking system of cell culture, comprising:
a shaking unit;
a side visual unit, located at one side of the shaking unit;
a top visual unit, located above the shaking unit; and
a control unit, connected signally with the shaking unit, the side visual unit and the top visual unit.

2. The shaking system of cell culture of claim 1, wherein the shaking unit has a variable eccentric-rotating module, a linkage module and a variable center-rotating module, the variable eccentric-rotating module being coupled with the linkage module, the linkage module being coupled with the variable center-rotating module.

3. The shaking system of cell culture of claim 2, wherein the variable eccentric-rotating module has a drive gear, a transmission shaft, an eccentric cam, an eccentric slider, an adjusting member and a first spherical bearing, the transmission shaft being coupled with the drive gear, the eccentric cam being coupled with the transmission shaft, the eccentric slider being located inside the eccentric cam, the adjusting member being coupled with the eccentric slider, the first spherical bearing being coupled with the eccentric slider.

4. The shaking system of cell culture of claim 3, wherein the linkage module has a connecting rod, an auxiliary guide bar, a second spherical bearing and a third spherical bearing, the connecting rod being coupled with the first spherical bearing, one end of the auxiliary guide bar being coupled with the connecting rod while another end thereof is coupled with the second spherical bearing, the third spherical bearing being coupled with one end of the connecting rod.

5. The shaking system of cell culture of claim 4, wherein the variable center-rotating module has a base frame, a slider, at least one guide bar, a screw bar, a carrier board, the base frame being coupled with the third spherical bearing, the slider being located on the base frame, the guide bar and the screw bar being individually sent through the base frame and the slider, the screw bar being coupled with a power source, the carrier board being fixed to the slider and having four positioning pins standing individually at four respective corners thereof.

6. The shaking system of cell culture of claim 5, wherein the third spherical bearing is coupled with the base frame.

7. The shaking system of cell culture of claim 5, wherein the power source is one of a motor, a gear set, a screw bar, a pneumatic cylinder and a hydraulic cylinder.

8. The shaking system of cell culture of claim 5, wherein the screw bar drives the slider to displace with respect to the base frame, such that the variable center-rotating module can vary a position of a rotational center point.

9. The shaking system of cell culture of claim 1, wherein the top visual unit and the side visual unit are selected from the group of charge coupled devices (CCD), digital cameras and digital recorders.

10. An shaking method of cell culture, comprising the steps of:
shaking a cell-culture dish, which is furnished in shaking system and contains a kind of culture fluid, at a fixed rotation speed within a small angular range;
acquiring an image information of the cell-culture dish by a top visual unit;
acquiring the distribution area of the culture fluid by a control unit in accordance with the image information;
performing rotationally shaking toward the non-liquid surface distribution area in accordance with the distribution area;
changing the shaking speed and the shaking angle by the shaking system to make the culture fluid distributed in the non-liquid surface distribution area;
acquiring the image information of the culture liquid surface height of the cell-culture dish by a side visual unit;
changing the shaking angle by the control unit in accordance with the image information of the liquid surface height to increase or decrease the shaking angle;
judging if a 100% culture medium coverage rate has been achieved;
acquiring again the image information of the cell-culture dish by the top visual unit and the side visual unit;
confirming the distribution status of the culture liquid by the control unit in accordance with an image information history.

11. The shaking method of cell culture of claim 10, wherein the fixed rotation speed is 9.5 rpm, and the small angular range is a range of ±5°.

12. The shaking method of cell culture of claim 10, wherein, in the step of acquiring the image, the control unit receives the top-viewed image information, determines a region of interest from the top-viewed image information, then transforms the region of interest into a gray-scale image, further strengthens the gray-scale image so as to transform the gray-scale image into a binarized image, and then performs a noise-filtration process upon the binarized image so as to form a processed image; wherein the processed image is used to calculate the distribution of the culture fluid.

13. The shaking method of cell culture of claim 10, in the step of judging whether or not the 100% culture medium coverage has been achieved, keeping shaking and going back to the step of basing on the distribution to shake rotationally toward the uncovered area of the distribution if a judgment thereof is negative, stopping shaking if the judgment is positive.

14. The shaking method of cell culture of claim 10, wherein, in the step of basing on the distribution to shake rotationally toward the uncovered area of the distribution, when the angular range of shaking is enlarged, the shaking system of cell culture changes a position of a rotational center point.

15. The shaking method of cell culture of claim 10, wherein, in the step of basing on the liquid level height to increase or decrease the shaking angle, the control unit receives the side-viewed image information, determines a region of interest from the side-viewed image information, then transforms the region of interest into a gray-scale image, further strengthens the gray-scale image so as to transform the gray-scale image into a binarized image, and then performs a noise-filtration process upon the binarized image so as to form a processed image; wherein the processed image is used to calculate the liquid level height of the culture fluid.

16. The shaking method of cell culture of claim 15, wherein, in the step of judging whether or not the 100% culture medium coverage has been achieved, a control unit bases on all changes of the individual gray-values in all the image information to confirm the distribution of the culture fluid and to obtain a computed coverage of the culture fluid, and further bases on the computed coverage to determine whether or not the shaking performed by the shaking system shall be proceeded, proceeding the shaking and going back to the step of basing on the distribution to shake rotationally toward the uncovered area of the distribution if a determination thereof is negative, stopping the shaking if the determination is positive.

17. The shaking method of cell culture of claim 10, wherein, in the step of shaking at the fixed rotation speed within the small angular range, the cell-culture dish is arranged on a variable center-rotating module of the shaking system, a variable eccentric-rotating module of the shaking system drives the variable center-rotating module to shake the cell-culture dish at the fixed rotation speed within the small angular range.

18. The shaking method of cell culture of claim 17, wherein, in the step of basing on the liquid level height to increase or decrease the shaking angle, a slider of the variable center-rotating module displaces with respect to the base frame of the variable center-rotating module so as to vary the shaking angle for enlarging/narrowing the angular range of shaking.

19. The shaking method of cell culture of claim 17, wherein, in the step of basing on the distribution to shake rotationally toward the uncovered area of the distribution, a slider of the variable center-rotating module displaces with respect to the base frame of the variable center-rotating module so as to vary the shaking angle.

20. The shaking method of cell culture of claim 17, wherein, in the step of shaking at the fixed rotation speed within the small angular range, a power source drives the variable eccentric-rotating module by the fixed rotation speed, then the variable eccentric-rotating module drives a linkage module, and then the linkage module drives the variable center-rotating module, so that the variable eccentric-rotating module shakes the variable center-rotating module within the small angular range.
